(19) United States
(12) Patent Application Publication
(10) Pub. No.: US 2016/0143686 A1
(43) Pub. Date: May 26, 2016

(54) INTER-ELECTRODE IMPEDANCE FOR DETECTING TISSUE DISTANCE, ORIENTATION, CONTACT AND CONTACT QUALITY

(71) Applicant: STEREOTAXIS, INC., St. Louis, MO (US)

(72) Inventors: Ilker Tunay, St. Louis, MO (US); Nathan Kastelein, St. Louis, MO (US)

(21) Appl. No.: 14/946,767

(22) Filed: Nov. 19, 2015

Related U.S. Application Data
(60) Provisional application No. 62/082,120, filed on Nov. 19, 2014.

(51) Int. Cl. A61B 18/12; A61B 18/14

(52) U.S. Cl. CPC A61B 18/1233; A61B 18/1492; A61B 2018/00011

(57) ABSTRACT

A method of determining the distance between an electrode catheter disposed in a body fluid adjacent an internal body surface, and the internal body surface, the method comprising: applying an alternating voltage or an alternating current that alternates at between about 10 kHZ and about 100 kHz between at least one pair of electrodes on the electrode catheter; determining the impedance between at least one pair of electrodes on the electrode catheter; and determining the distance between the electrode catheter and the internal body surface based at least in part on the determined impedance.

### CROSS-REFERENCED APPLICATION

This application claims priority to U.S. provisional application Ser. No. 62/082,120 filed on Nov. 19, 2014. The disclosure of the above-referenced application is incorporated herein by reference in its entirety.

### FIELD

The present disclosure relates to the use of inter-electrode impedance for detecting tissue distance, orientation and existence and quality of contact between a medical device and tissue.

### BACKGROUND

This section provides background information related to the present disclosure which is not necessarily prior art.

The present disclosure relates to detecting distances and orientations of tissue walls relative to electrophysiology catheters inside body lumens for medical applications, and existence and quality of contact between the catheter tip and tissue. The exemplary medical applications may include detecting endocardial and epicardial tissues, detecting endovascular tissues and occlusions, and other parts of the anatomy where interfaces exist between bulk liquids (blood, lymph, water and digestive fluids) and tissues having different structures than the liquids, such as myocardium, stomach lining, etc.

Detected distances, orientations, contact and quality of contact are useful for navigating catheters in body lumens, making tip contacts with tissues, estimating tissue contact forces, and assessing contact quality and stability in real-time as tissues move and for improving contact quality using automated algorithms or helping a human operator towards a diagnostic or therapeutic goal. The distance is understood to be negative when the tip is in contact with the tissue, and positive when it is not in contact. Applying a force to the tissue surface by pushing the device tip further into the tissue will cause surface deformation. Therefore, in this context distance means the distance to the undeformed surface, and a negative distance means that the device tip has been advanced beyond the undeformed surface.

Conventionally, impedance measurements for detecting tissue contacts or distances to tissues are used between tip electrodes of electrophysiology catheters and dispersive electrodes placed externally to the body. These are commonly called unipolar measurements. Such measurements are often affected by many factors, e.g., device motions or changes in thoracic impedance due to respiration, patient torso deformations and/or shifting of the dispersive surface patches caused by gross patient motions, and variations in skin-patch interface impedance, etc. Accordingly, these measurements are usually not useful for reliable contact sensing.

### SUMMARY

This section provides a general summary of the disclosure, and is not a comprehensive disclosure of its full scope or all of its features.

Embodiments of the present disclosure provide methods for detecting tissue distances from catheter tips, including negative distances in which contact is present, orientation of tissue surface relative to the catheter tip direction, existence of contact and quality of contact in the sense that a substantial percentage of the tip surface remains in contact with the tissue during cardiac and respiratory cycles in the face of tissue motion and blood flow, using local nature of currents in-between catheter electrodes located in close proximity to each other and near the distal tip of the catheter. When one pair of electrodes is used, with one electrode that acts as a current source and another one that acts as a sink, the measurement is commonly known as bipolar. However, the methods described here are applicable to impedance between more than one pair of electrodes and more than one sink electrode for every source electrode. Furthermore, information is gained from both components of the complex impedance whose equivalent descriptions include a resistance and a reactance (or capacitance), or a real and an imaginary part, or a magnitude and phase angle. Such detecting methods are generally independent of the navigation or actuation means of the catheters, whether the catheter is manipulated by an operator or automatically by software, or whether the operator or software are located remotely from the patient. Moreover, unipolar impedance measurements may be combined with one or more bipolar measurements. These measurements are input to an algorithm which may also collect status information from various other devices being used for the procedure, such as an RF generator, irrigation pump, ECG monitor, blood pressure monitor, etc. and which has information about the particular body chamber in which the catheter is being navigated, such as one of the heart chambers. When magnetic navigation is being used to direct the catheter, the difference between the orientation of the applied magnetic field and the catheter tip can be supplied to the algorithm as additional information to be used in determining contact. The algorithm may include adaptive or machine learning features so that it may improve itself during a procedure in one patient or by collecting information from procedures in multiple patients and analyzing those while off-line.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1D are schematic diagrams illustrating different contacts and orientations between a distal tip of an electrode and the surface of the myocardium;

FIG. 2 is a schematic diagram of the distal end of an electrode catheter showing the current density between a tip electrode and a second ring electrode;

FIG. 3 is a chart showing impedance between the tip and the second ring electrode, with its baseline blood value subtracted, at various tip distances before surface contact;

FIG. 4 is a chart showing impedance between the tip and the second ring electrode, with its baseline blood value subtracted, at various angular tip orientations before contract;

FIG. 5 is a chart showing impedance between the tip and a second ring electrode, with its baseline blood value subtracted at various tip distances after surface contact;

FIG. 6 is a chart showing impedance between the tip and a second ring electrode, with its baseline blood value subtracted at various angular tip orientations after contact;

FIGS. 7-10 are graphs showing different bench results with different contact situations between a catheter and a surface;

FIGS. 11A-11H are schematic diagrams showing the different impedance measurements resulting from different contact scenarios;

FIG. 12 is flow chart of a real time multi-channel impedance measurement system;

FIG. 13 is a four terminal circuit diagram;

FIG. 14 is a Mod/Demod circuit diagram;

FIG. 15 is an in vivo unipolar impedance chart in a blood pool;

FIG. 16 is an in vivo unipolar impedance chart adjacent the LA lateral wall;

FIG. 17 is an in vivo unipolar impedance chart showing the transition from contact with LA lateral wall to blood pool;

FIG. 18 is an in vivo unipolar impedance chart showing the transition from within a pulmonary vein to its ostium and back; PV->Ostium->PV;

FIG. 19 is an in vivo two channel bipolar impedance chart in the blood pool;

FIG. 20 is an in vivo two channel bipolar impedance chart in tva pulmonary vein;

FIG. 21 is an in vivo two channel bipolar impedance chart showing the transition from within blood pool to vertical contact with the mitral wall;

FIG. 22 is an in vivo two channel bipolar impedance chart showing the transition from within blood pool to parallel contact with the mitral wall; and

FIG. 23 is a schematics diagram of the operation of possible classifiers suitable for use with any of the embodiments of this invention.

Corresponding reference numerals indicate corresponding parts throughout the several views of the drawings.

### DETAILED DESCRIPTION

Example embodiments will now be described more fully with reference to the accompanying drawings.

The detecting methods can apply to both manually and automatically (e.g., magnetically or robotically) steered catheters. An electrode that is used to inject current into the environment is called a source electrode. An electrode that is used to collect current is called a sink electrode. For example, in a preferred embodiment a catheter tip with four electrodes, numbered from distal to proximal, tip (1) and first ring (2) electrodes can be used as sources; second ring (3) and third ring (4) electrodes can be used as sinks. Alternatively, or simultaneously with the above pairing, using time or frequency multiplexing, one may designate any one of the four electrodes as source and the other three as sinks. For instance, assigning electrode 1 as source and electrodes 2, 3 and 4 as sinks is useful for resolving cases in which complicated tissue structures such as trabeculations alter the resulting local distribution of current density.

For devices which are supported with a sheath from which they are extended into the local anatomical structure, it can be useful to know the moment when the device tip, or a landmark near the tip, such as an electrode, is exiting the sheath or is being retracted into the sheath. This information can be used to calibrate sheath position and orientation (posture), provided that the catheter has a location sensor operating independently from the electrodes. This information can also be used to calibrate extended catheter length. Sheath posture and extended length are important inputs to mechanical models of catheter behavior. When the electrodes are passing from the sheath into the surrounding blood or vice versa, local current density will change, often dramatically. The measured impedance between a pair of electrodes will increase rapidly to a very high value when one or both electrodes of the pair retract into the sheath; it will decrease rapidly to its normal range when both electrodes exit the sheath. Depending upon the total number of electrodes in a group of adjacent electrodes, only a small number of discrete states exist, each such state corresponding to a specific subset of electrodes being disposed inside the sheath. Transition from any one state to another will correspond to a specific sequence of impedance changes or “jumps”. Starting from a known initial state, such as all electrodes being inside or outside the sheath, and matching the pattern of observed impedance jumps to known set of patterns, one can determine unambiguously which state transition has taken place and thus the current state.

Pattern matching techniques, which are invariant to time scaling, are preferred so that the speed of the catheter motion will not alter the match. A library of patterns can be built from finite-element simulations, by bench tests using a fluid whose conductivity approximates a particular body fluid, by in vivo animal tests within the particular body chamber, or calculation or empirical methods.

For an axially symmetric device, such as a catheter, ring electrodes placed along the shaft near the distal tip are appropriate. For a device which is non-axially symmetric, one may use patch electrodes which are placed on one side of the device shaft in addition to or in place of ring electrodes. Such a construction can provide additional information about relative orientation of tissue.

Adaptive parameter estimation algorithms can be employed to build a local tissue surface representation and to construct a learning classifier to decide, in real-time, between adequate contact and non-contact. Local conductivity of blood is influenced by several factors including blood flow velocity, hematocrit percentage, and the rate of open irrigation with saline. The volumetric irrigation rate is known, for example from the irrigation pump. The patient's heart rate is also known, for example from the ECG system or from a catheter tip electrograms, which allows calculation of the average volumetric blood flow rate around the catheter when combined with the knowledge of approximate anatomical location of the catheter tip, such as within a heart chamber.

When the catheter tip is exiting the sheath but is not in contact with tissue, a few impedance measurements can be performed to calibrate blood conductivity of the particular patient. Thus, the fluid conductivity value used in the algorithms can be adjusted during the intervention.

Impedance at a particular frequency is a complex number which has a magnitude and a phase or equivalently, resistance and reactance, or real and imaginary components. These components can be measured by applying an alternating voltage between the electrodes and measuring the current, or alternatively, in a preferred embodiment, applying an alternating current between the electrodes and measuring the voltage drop. In the latter method, the current applied to the body is limited to a small value, such as 100 microAmperes, regardless of the total impedance between electrodes and thereby is safer. Typical signal frequencies are between about 5 kHz and about 200 kHz. This range discriminates well between blood and tissue; it has the potential to identify blood impedance dispersion due to the change in the impedance component of red blood cell membrane capacitance; and is sufficiently separated from typical RF ablation frequency of 500 kHz. More preferably the signal frequency (whether applied voltage or applied AC current) is between about 10 kHz and about 100 kHz, and most preferably it is about 20 kHz.

The applied current amplitude is kept small in the fractional mA range. The electronics can be designed so that it does not interfere with ECG measurements, RF ablation currents or other sensitive biomedical instrumentation and is protected from defibrillator voltages. By using frequency separation, or differential op-amp circuits, or time domain switching or other electronic means, the impedance between two pairs of electrodes can be measured almost simultaneously and independently. In a preferred embodiment, two sinusoidal signals whose frequencies are separated by at least a few kilohertz are injected. Synchronous demodulation followed by low-pass filtering is used to isolate one of the injected signals from background noise and also from the other injected signal. A reference signal in phase with the injected signal can be used to perform the resistance measurement. Another reference signal with 90 degrees phase difference with the injected signal can be used to perform the reactance measurement.

In the frequency range of interest, reactance of both the blood and tissue is capacitive. Those reactance values are highly sensitive to the frequency of excitation in the 5 to 200 kHz range since cell membranes become more conductive with increasing frequency. However, it is expected that the dispersion of tissue conductivity will be different from that of blood. By performing a frequency sweep or by injecting a multitude of frequencies one after the other and making decisions based on the aggregate information, it may be possible to improve the contact detection.

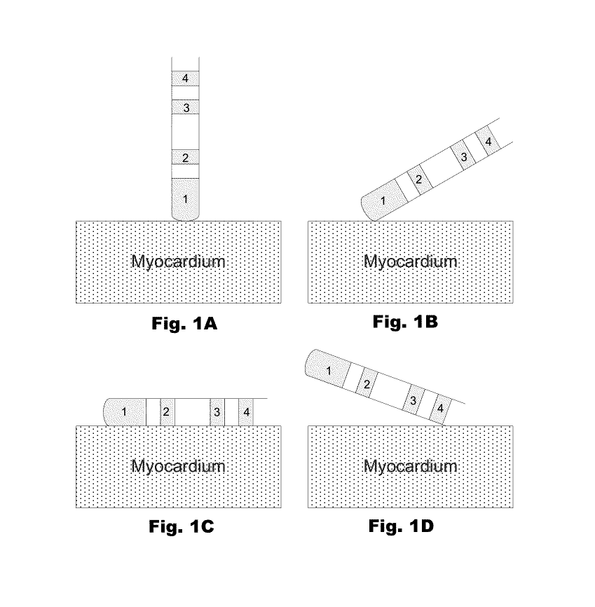

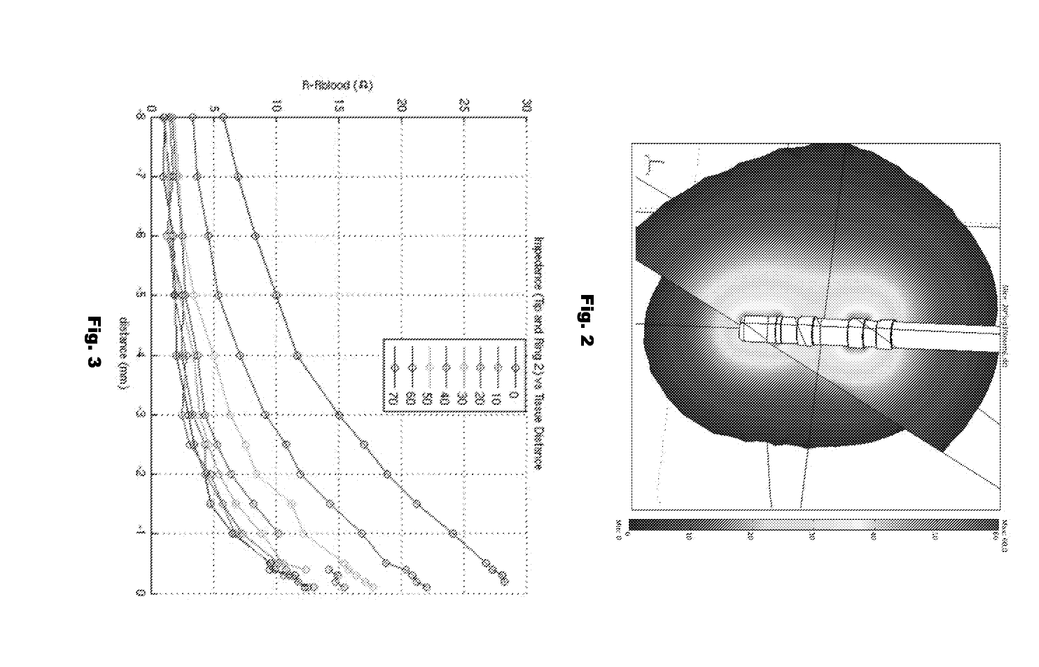

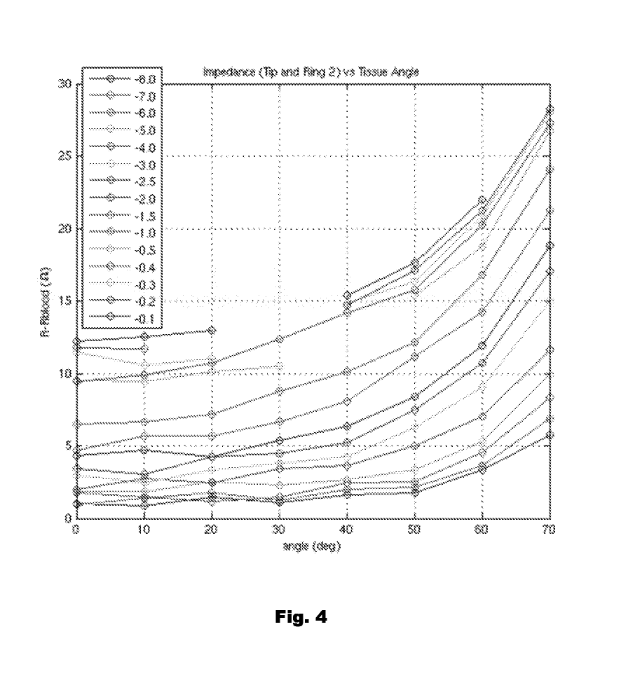

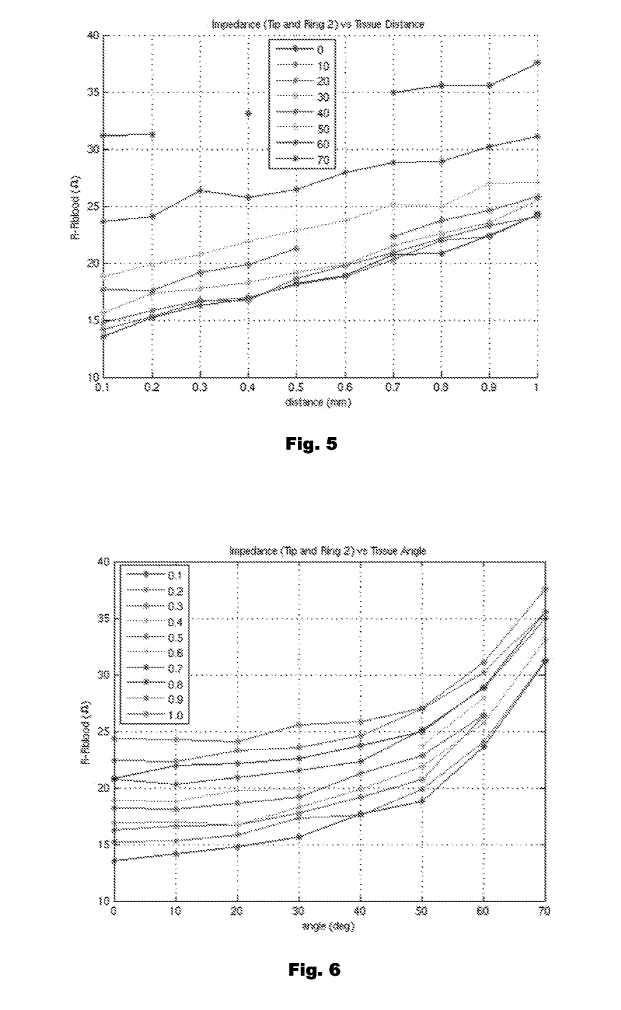

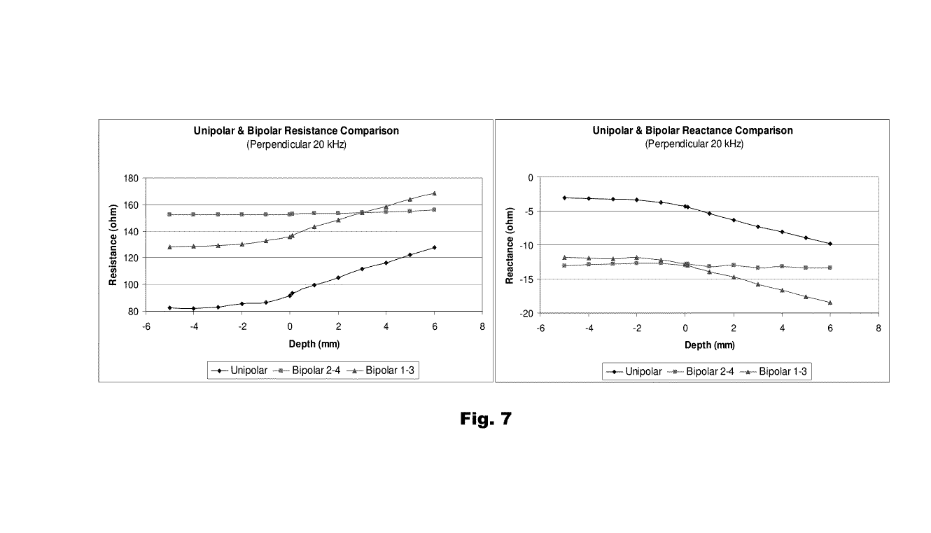

FIG. 1 shows a catheter with a tip electrode and three ring electrodes in the vicinity of tissue, inside blood. Three-dimensional finite-element analysis (3D FEA) in FIG. 2 shows that the current is localized to the vicinity of catheter tip. FIGS. 3 and 5 show that impedance grows quadratically before contact, and grows linearly after contact. FIGS. 4 and 6 indicate that impedance is also sensitive to tissue orientation relative to the catheter axis.

If Z1 and Z2 denote the impedance between electrodes 1-3 and 2-4 respectively, where the electrodes are numbered from distal to proximal. And if x denotes the perpendicular distance from the local tissue surface patch to the catheter tip, and θ denotes the angle between the catheter tip axis and the normal vector of the tissue patch. The forward functional relationships Z1=f1(x, θ) and Z2=f2(x, θ) are continuous and monotonic in their arguments and thus are locally invertible to give the inverse functional relationships x=g1(Z1,Z2) and θ=g2(Z1,Z2). The forward functions f1 and f2 can be represented with polynomials, piecewise polynomials, Legendre polynomials, feed-forward neural networks or combinations of other differentiable basis functions. If quadratic or cubic polynomials yield a good fit to the data, the inverse can be found analytically. Otherwise, an optimization or search algorithm can be used to find the local minimizer (x, θ) of the cost function (Z1−f1(x, θ))²+(Z2−f2(x, θ))². Bisection search, damped Newton or other differentiable unconstrained optimization algorithms are also appropriate.

Open irrigation using saline or other physiologically compatible fluid from the tip electrode of a catheter is commonly employed to prevent or reduce the possibility of formation of blood clots and their adherence to the tip electrode. Such irrigation also performs a cooling function in the vicinity of the tip so that the radiofrequency energy being delivered results in a more even temperature rise inside the tissue. A sudden and drastic change in the irrigation rate, which is commonly used just prior to RF energy application, results in a drastic and measurable change of a few nanoFarads in the capacitance (or equivalently, reactance) component of the impedance only for the pair of electrodes that includes the tip electrode, but does not affect the resistance component significantly. By observing this drastic change in capacitance, one may conclude that irrigation has started and also by using a look-up table, may be able to estimate the irrigation rate. Such information is useful for adjusting the contact detection algorithm by observing the impedance just prior to RF application.

For the pairs of electrodes being used for contact detection, the baseline impedance values which correspond to the catheter being inside a chamber filled with body fluid but spaced from tissue walls is an important input to the following algorithms. These baseline values can change during the course of a procedure since external fluids are commonly administered to a patient, respiration and perspiration result in water loss and kidneys remove excessive water and salts from the blood, therefore changing the conductivity of body fluids, and blood in particular. However, such change occurs slowly and gradually compared to navigation motions done by the catheter. One way to determine the current baseline value for a particular pair of electrodes is to identify the moment when that pair is exiting from a sheath into a chamber and to use that impedance measurement as the new baseline. Another way to determine the current baseline is to measure the conductivity of the body fluid and blood in particular, by placing a separate reference catheter into the same or another chamber or large vessel containing the same body fluid and measuring impedance between pairs of electrodes on that catheter.

It is not necessary that the reference catheter have the same number, shape, size or spacing of the electrodes as the catheter being navigated. For instance, during cardiac procedures, a reference catheter may be placed into a vena cava or an atrium, away from tissue walls and secured so that cardiac or respiratory motion does not affect its position significantly during the whole procedure. The approximate dimensions of the chamber or vein will be known for the species under operation. A geometrical computer model of that chamber or vein may be built and the finite-element method may be used to compute inter-electrode impedances for a geometrical model of the particular reference catheter in that chamber. Since the electrical current density drops quickly with the distance from the electrodes and the temperature of the patient is fairly constant, the resistance between a pair will depend primarily on the conductivity of the fluid. A look-up table may be prepared which will describe the functional relationship between conductivity and computed resistance for each pair of electrodes.

During a procedure, using the measured resistance as the input, one may interpolate the table to determine the current value of fluid conductivity. A similar table can be prepared by finite-element simulations of the navigation catheter in a large chamber. During a procedure, using the determined fluid conductivity as the input, one may interpolate the second table to determine the baseline resistance between a pair of electrodes. The same procedure can be repeated for baseline capacitance values by using fluid permittivity in addition to conductivity.

Another method to determine baseline values is to extract a small amount of fluid from the patient at certain intervals and measure its conductivity using common laboratory equipment and techniques. This measured conductivity may be used as the input to the second lookup table above to output baseline resistances.

Yet another method to determine baseline values in blood is to build a physiological model of the concentration of salts in a patient's blood. Given a patient's body weight and recent history of fluid intake, the total volume of blood in the body is known approximately. The change in the concentration of salts in the blood, and in particular, that of sodium chloride can be computed approximately from a model using the known rate of intravenous fluid administration, fluid ingestion, amount of irrigation from the catheter and that patient's kidney competence. The estimated salt concentration can be used to compute blood conductivity by referring to data in medical literature. This or some other computed conductivity may be used as the input to the second table above to output baseline resistances.

It is useful to know approximately the contact force between the tip of the catheter and tissue walls for the purpose of avoiding excessive tissue deformation or perforation. The correspondence between impedance of electrode pairs and contact force will depend on tissue surface smoothness, amount of trabeculation, presence of scar or previous lesions, all of which influence local compliance of tissue. By using a force-sensing catheter which is also equipped with pairs of electrodes near its tip during in-vivo experiments with multiple subjects, it is possible to build a library of tissue types which contain the functional relationship between the normal and tangential components of the contact force and the position and orientation of the catheter tip relative to the undeformed tissue surface. Using a look-up table or polynomial fitting or any other mathematical function approximation, the components of the contact force may be expressed as functions of the catheter position and orientation. During a procedure, the measured inter-electrode impedances can be employed to compute the catheter position and orientation using one of the algorithms described below and those variables will be inputs to the said function approximation methods, yielding an approximate estimate of the contact force.

During a medical therapeutic procedure which uses a catheter to induce thermal effects into the tissue, such as radiofrequency ablation of cardiac muscle, the temperature of the tissue in the vicinity of the catheter, and to a lesser extent, that of the blood, will increase. This will change (increase) the conductivity of the tissue and blood, therefore decreasing the resistance measured between pairs of electrodes. It is useful to compensate for these thermally-induced changes in measured resistances since determination of tissue contact and relative position and orientation of the catheter depend on them. One way to perform this compensation is to measure the temperature of the tip electrode with a thermocouple or other sensor, which is commonly found in ablation catheters. Then, one may use the data (for example available existing literature) which correlates conductivity with temperature. However, irrigation of the tip using room-temperature saline or other fluid will alter the temperature measurement significantly. Another way to perform this temperature compensation is to use the unipolar impedance from the tip electrode to a dispersive electrode, usually placed on the skin of the patient. Since thermally-induced conductivity changes will influence unipolar impedance in the same manner as bipolar impedance, the relative change in the unipolar impedance may be used as a correction factor for bipolar impedance. A further refinement of this method is to use a thermal-electrical finite-element model of the catheter, blood and tissue to compute both unipolar and bipolar resistance values at various catheter distance and orientation combinations while the heat addition due to radiofrequency or microwave energy application and heat removal due to blood flow, irrigation and perfusion are taking place. Using the known power setting of the ablation generator and the anatomical location of the tissue and heart rate, hence blood flow rate in the vicinity of that tissue, the relative change in the unipolar impedance will show a better correspondence to the bipolar values.

Four exemplary algorithms are provided: The first one is applicable with or without a catheter localization system (e.g., commercially available Carto or NavX systems) and gives an instantaneous estimate of the tissue distance x and angle θ. The second one requires a localization system and multiple impedance readings but gives an estimate of the location and shape of the tissue patch in a global coordinate frame.

Algorithm A: Tissue Distance and Orientation Estimation and Contact Existence and Quality Detection

(a) Using results from 3D FEA and ex-vivo experiments, a functional model can be developed that estimates impedance between two pairs of electrodes as a function of tissue distance and angle.

(b) During a medical procedure, while all electrodes are in the blood pool away from a tissue surface, calibrating the functional model by using blood values of Z1 and Z2 as baseline. Using the known chamber information to include the effect of blood flow rate and turbulence in the calibration.

(c) As new impedance measurements are obtained every measurement period (e.g., 10 ms), optimization or search algorithm is used to solve the above minimization problem to find the best estimates of tissue angle and distance, including negative distance which indicates tip penetration or tissue deformation, and to decide whether contact has occurred. The recent history of impedance measurements and known catheter actuation values, such as changes in inserted length or orientation, together with known cardiac cycle phase from ECG data, are used to correlate impedance history to catheter to tissue distance history.

(d) Contact quality can be determined from tissue distance and angle change within a cardiac cycle. Furthermore, during a cardiac cycle, the amplitude of the variation in the resistance components relative to those of reactance components, and the difference in variations in Z1 and Z2, can be used to estimate the percentage of tip (1^{st}) electrode surface in contact with tissue surface, which is a measure of contact quality.

(e) If open irrigation is present, the functional model can be adjusted according to precomputed conductivity values for the known irrigation rate and measured heart rate. If RF energy is being applied to tissue for therapeutic purposes, the functional model can be adjusted to account for the change in conductivity and permittivity values of tissue due to temperature increase and to destruction of cellular structure within the myocardium.

Algorithm B: Tissue Patch Location and Shape Estimation

(a) The local tissue surface can be represented with a parametric model. For a linear model, distance and two angles for orientation are used. For a quadratic model, distance, two curvatures for shape, and two angles for orientation are used.

(b) Using results from 3D FEA and ex-vivo experiments, a functional model that estimates impedance between two pairs of electrodes as a function of surface parameters can be developed.

(c) A functional model that relates impedance to contact force can also be developed.

(d) During a medical operation, as the catheter approaches an anatomic location inside the lumen, initial local surface parameters from previous localization measurements (e.g. a Carto or NavX map) or previous 3D imaging (e.g. CT or MRI scan) are estimated. If none is available, default parameters can be used.

(e) During a medical operation, while all electrodes are in the blood pool away from a tissue surface, the functional model can be calibrated by using blood values of Z1 and Z2 as the baseline.

(f) Using the previously developed functional model, and a recursive, on-line parameter identification algorithm such as least-squares or maximum likelihood, the parameters are updated as new impedance measurements are obtained every measurement period (e.g., 10 ms).

(g) If necessary for unambiguous estimation, the catheter position and orientation can be quickly changed a few times to generate more data.

(h) As the catheter makes contact with the tissue, the parameters can be updated as necessary.

(i) Contact force using the previously developed functional model.

(j) Contact stability can be determined from estimates of contact force, tissue distance and orientation change within a cardiac cycle.

(k) If open irrigation is present, the functional model can be adjusted according to precomputed conductivity values for the known irrigation rate and measured heart rate.

Algorithm C: Use of a Classifier System to Assess Contact

(a) Through evaluation of data from multiple procedures, key parameters including one or more channels of bipolar impedance, unipolar tip impedance, tip temperature, ablation generator state and catheter navigation state can be used to train a classifier system such as an artificial neural network to recognize the conditions associated with catheter to tissue contact. Through selection of suitable training examples and suitable training, the classifier would be able to distinguish contact vs non-contact in normal tissue contact situations and during contact situations where the tissue characteristics are changing due to the application of RF energy.

(b) Preparation of the inputs to the classifier system can be important part of how the system functions. Because the body, especially the heart, is a dynamic environment, aggregation of the data over a short sampling period (3 seconds for example) provides a clearer picture of the overall state of contact. Short term sampling of the signal amplitude and signal baseline of the each of the bipolar measures, resistance and reactance for distal and proximal electrodes provides a set of the key inputs to the classifier. In addition, the signal has a periodic nature due to the changes in contact caused by the heartbeat and by respiration. Good contact is characterized by an increase in the periodic signal corresponding to the heart motion. A Fourier transform can provide a measure of the period frequency and provides the classifier with information which can be additionally used to assess contact.

(c) In a magnetically assisted procedure, the direction of the applied magnetic field and the direction of the catheter can provided additional detail concerning contact. A magnetic catheter, which is free to move, aligns itself with the prevailing magnetic field. A catheter which is obstructed, deviates from prevailing field due to the presence of tissue in the direction the catheter would normally be oriented. The amount of deviation between the catheter and the prevailing field indicates a build-up of forces which translate into catheter to tissue contact. The amount of deviation between catheter and navigation field can additionally be used by the classifier to assess contact.

(d) The application of RF energy to deliver therapy by ablating tissue can cause acute changes in the environment of the catheter tip. Temperature and tissue characteristics cause changes in the observed bipolar impedance. To allow a classifier system to compensate for these effects, the classifier ca additionally be provided with the on/off state of the RF generator, the unipolar impedance of the RF generator's electrical circuit, and the tip temperature. These ablation related parameters allow a classification system to assess contact in cases where RF energy is being delivered and in cases where it is not.

Algorithm D: Sheath Exit Detection

(a) A bench model of a heart chamber filled with a saline or other electrolyte solution whose conductivity is similar to that of blood and within which the solution is circulated at a velocity similar to the corresponding human heart chamber is used. Using the particular sheath and catheter, the catheter is placed at various relative positions within and extending from the sheath. Inter-electrode measurements are performed and stored. A finite Markov chain can be built whose states correspond to discrete values of catheter extension from the sheath. A landmark can be designated on the catheter shaft, such as one of the ring electrodes.

(b) Alternatively, a FE model of the sheath, catheter and fluid-filled chamber can be build and catheter extension simulated to collect and store the same information.

(c) During a procedure, the Markov chain is initialized with the known state of catheter being inside the sheath. Each time an impedance observation is made, the state based on the transition probability corresponding to the observation is updated.

(d) Each time it is known from independent sources of information that the catheter has been pulled into the sheath or that it is extended beyond the landmark, the Markov chain state is reset.

(e) When the exit of the landmark is detected, sheath posture and catheter length calibration are performed.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Specific dimensions, specific materials, and/or specific shapes disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

FIGS. 1A-1D are schematic diagrams showing different contacts and orientations between a distal tip of an electrode and the surface of the myocardium. FIG. 1A shows an electrode catheter oriented perpendicular to the surface of the myocardium, with distal tip in contact with the surface. FIG. 1B shows an electrode catheter oriented at an acute angle toward the surface of the myocardium, with the distal tip in contact with the surface. FIG. 1C shows an electrode catheter oriented parallel to the surface of the myocardium, with distal tip in contact with the surface. FIG. 1D shows an electrode catheter oriented at an acute angle away from the surface of the myocardium, with the distal tip not in contact with the surface.

FIG. 2 is a schematic diagram of the distal end of an electrode catheter showing the current density between a tip electrode and a second ring electrode on an electrode catheter,

FIG. 3 is a chart showing impedance between the tip and the second ring electrodes for a baseline (in blood) value, and various tip distances before surface contact. FIG. 3 illustrates that impedance values determined by electrodes on a device, particularly when compared to an appropriate base line value, can be of tip distance to a tissue surface.

FIG. 4 is a chart showing impedance between a the tip and a the second ring electrodes for the baseline (in blood) value and at various angular tip orientations. FIG. 4 illustrates that impedance values determined by electrodes on a device, particularly when compared to an appropriate base line value, can indicative of angular orientation of the device.

FIG. 5 is a chart showing impedance between the tip and a second ring electrode for a baseline (in blood) value and various tip distances after surface contact. FIG. 5 illustrates that impedance values determined by electrodes on a device, particularly when compared to an appropriate base line value, can indicative of distance from a tissue surface.

FIG. 6 is a chart showing impedance between the tip and a second ring electrode for the baseline (in blood) value and tip angles. FIG. 6 illustrates that impedance values determined by electrodes on a device, particularly when compared to an appropriate base line value, can indicative of angular orientation with respect to a tissue surface.

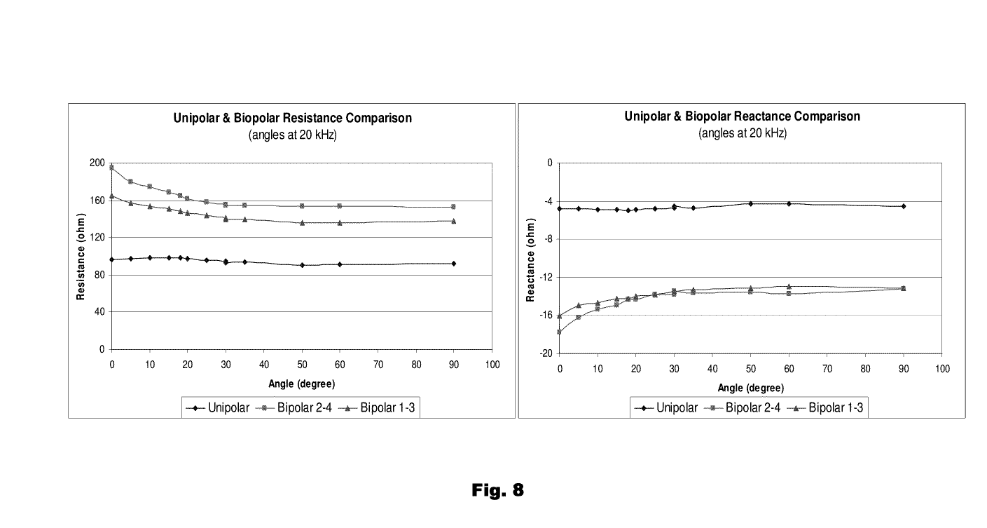

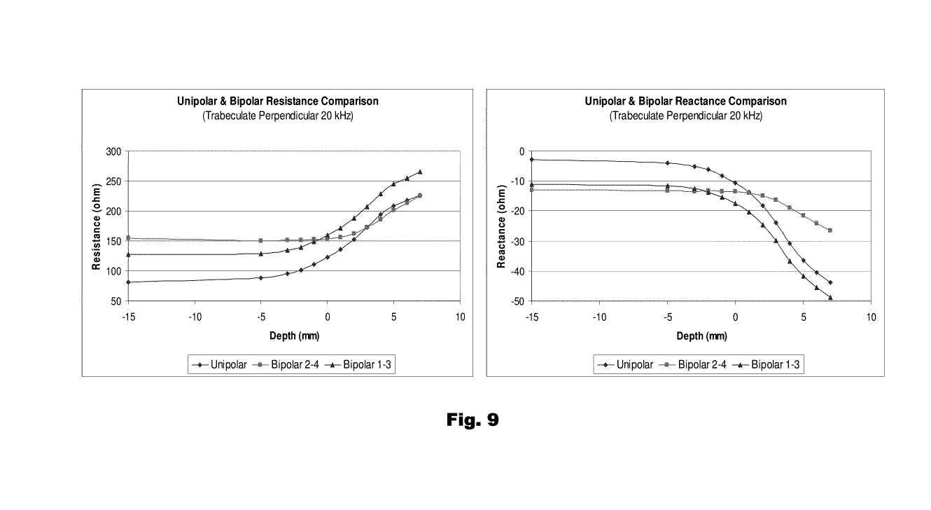

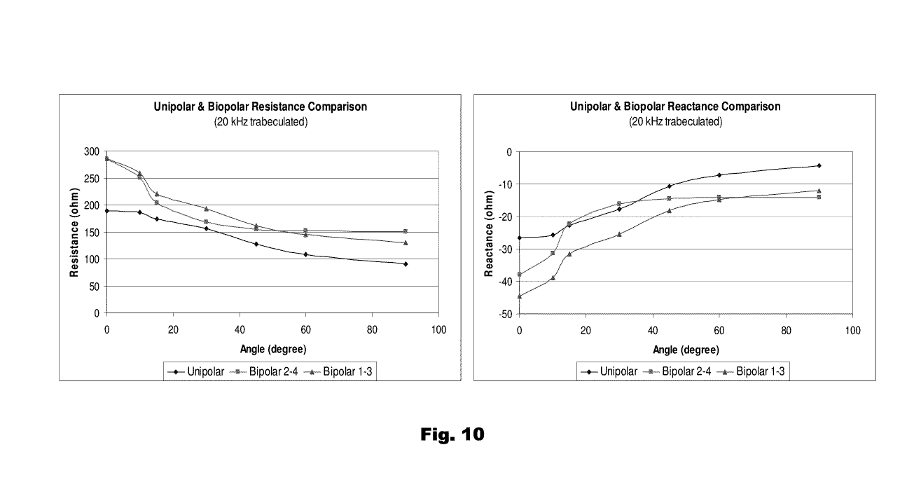

FIGS. 7-10 are graphs showing different bench results with different contact situations between a catheter and a surface. FIG. 7 shows that measurements of resistance and reactance vary with contact levels when the tip electrode is involved in the measurement, as it is with unipolar measurements and with measurements involving electrodes 1 and 3, but not when the tip electrode is not involved (e.g., the measurement between electrodes 2 and 4). FIG. 8 shows that measurements of resistance and reactance vary with angle (from 0° (parallel) to about 30°) of the device when using bipolar measurements, e.g. between electrodes 1 and 3 or 2 and 4, but not with a unipolar measurements involving only tip electrode 1. FIG. 9 shows that measurements of resistance and reactance vary with contact levels when the tip electrode is involved in the measurement, as it is with unipolar measurements and with measurements involving electrodes 1 and 3, but to a lesser degree when the tip electrode is not involved (the measurement between electrodes 2 and 4). FIG. 10 shows that measurements of resistance and reactance vary with angle (from 0° (parallel) to about 25°) of the device when using bipolar measurements, e.g. between electrodes 1 and 3 or 2 and 4, but not with a unipolar measurements involving only tip electrode 1.

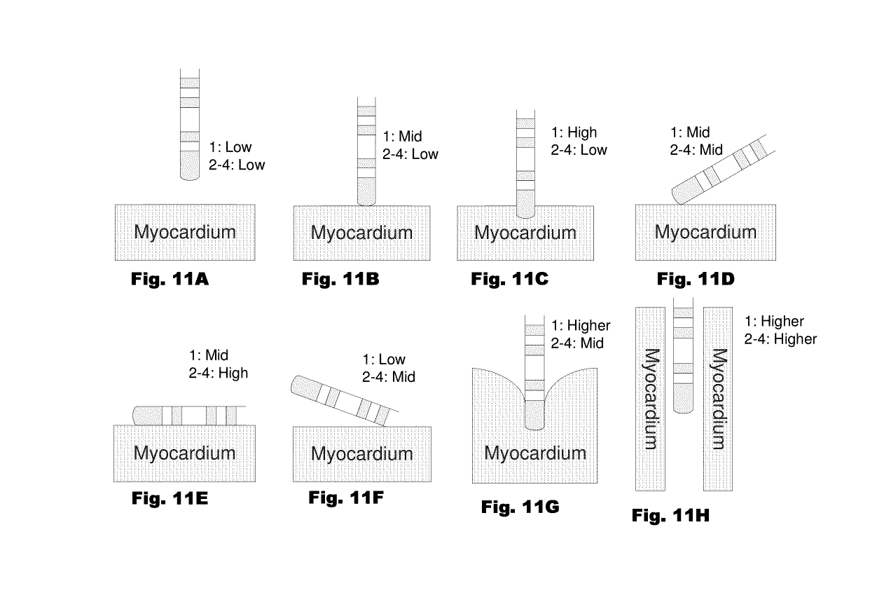

FIGS. 11A-11H are schematic diagrams showing the different impedance measurements resulting from different contact scenarios, and thus how these different measurements can be used to identify different contact scenarios. FIG. 11A shows that for a perpendicular orientation spaced from the tissue surface the unipolar impedance (1) and the bipolar impedance (2-4) measurements are low. FIG. 11B shows that for a perpendicular orientation with moderate surface contact, the unipolar impedance (1) measurement is intermediate, and the bipolar impedance (2-4) measurement is low. FIG. 11C shows that for a perpendicular orientation with significant surface contact, the unipolar impedance (1) measurement is high, and the bipolar impedance (2-4) measurement is low. FIG. 11D shows that for an accurate angle orientation toward the surface, with moderate surface contact, the unipolar impedance (1) and the bipolar impedance (2-4) measurements are moderate. FIG. 11E shows that for parallel orientation with surface contact, the unipolar impedance (1) measurement is moderate, and the bipolar impedance (2-4) measurement is high. FIG. 11F shows that for an angle orientation away from the surface, the unipolar impedance (1) measurement is low, and the bipolar impedance (2-4) measurement is low. FIG. 11G shows that for a perpendicular orientation with substantial surface contact, the unipolar impedance (1) measurement is higher (compared for example to FIG. 11), and the bipolar impedance (2-4) measurement is moderate. FIG. 11h shows that for a parallel orientation between two surfaces, the unipolar impedance (1) and the bipolar impedance (2-4) measurements are higher.

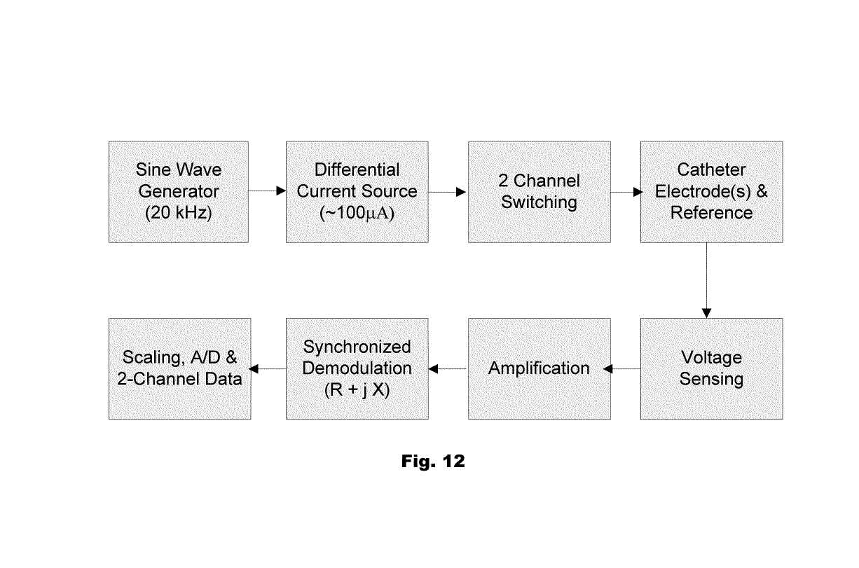

FIG. 12 is flow chart of a real time multi-channel impedance measurement system. A sine wave generator operating at between 5 and 200 kHz, and preferably at about 20 kHz as shown, and a differential current source (˜100 μA) and 2 channel switching are connected to the electrodes on a electrode catheter. The resulting voltages at the electrodes are sensed, amplified, and synchronized and demodulated. After scaling, bi-polar impedance values are available with which the orientation with respect to, and distance from, the surface can be determined.

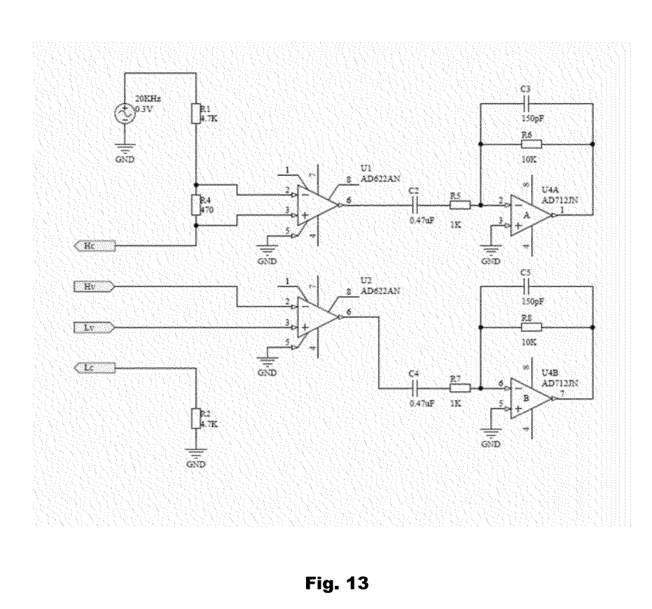

FIG. 13 is a four terminal circuit diagram of the type that could be used to capture bi-polar impedance measurements.

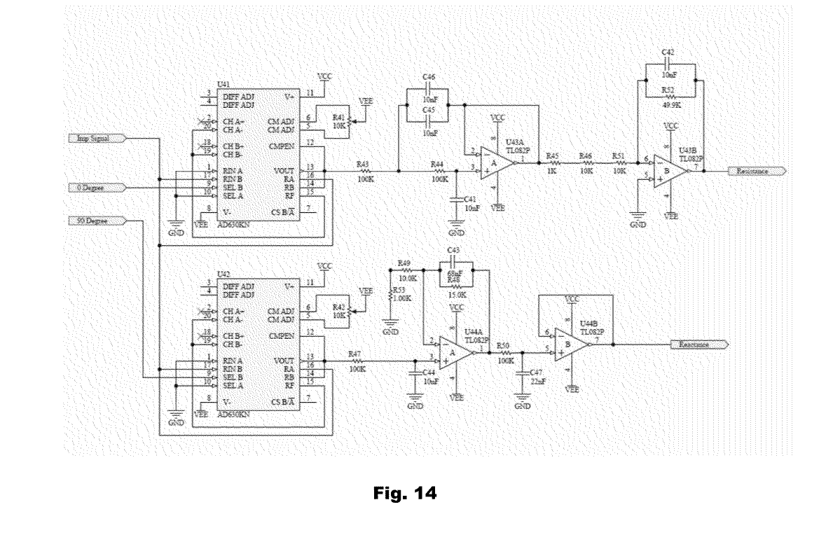

FIG. 14 is a mod/Demod circuit diagram that could be used to perform synchronized demodulation of the voltage signal to improve signal-to-noise ratio and also to extract resistance and reactance components of impedance.

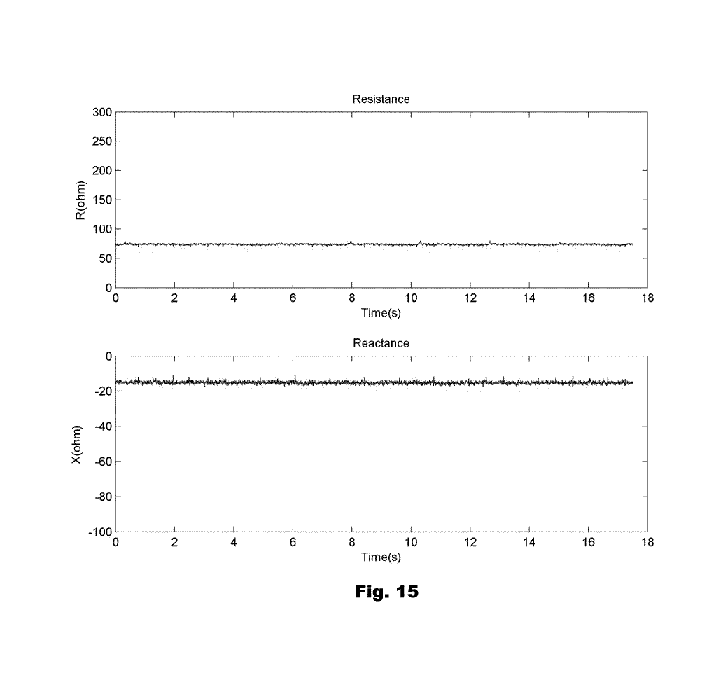

FIG. 15 is an in vivo unipolar resistance and impedance chart in a blood pool showing generally low, stable resistance, and generally low, stable impedance.

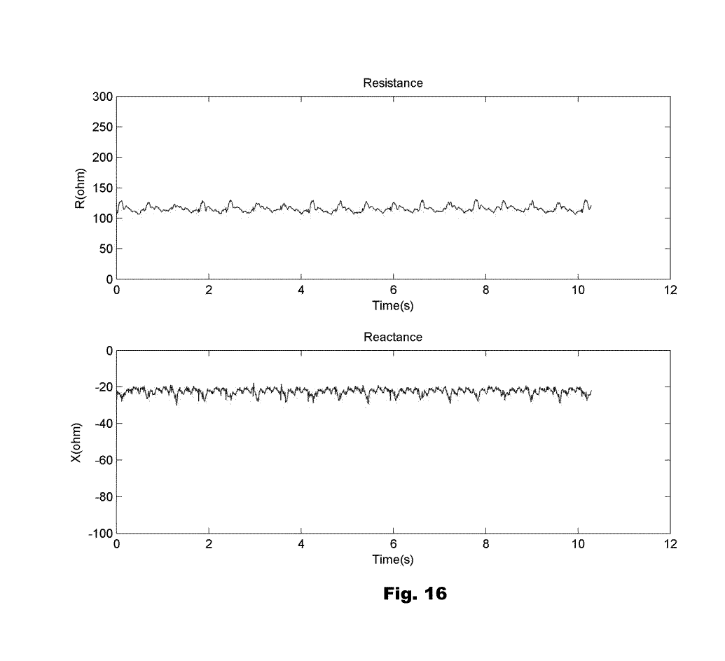

FIG. 16 is an in vivo unipolar resistance and impedance chart adjacent the LA lateral wall, showing generally high, stable resistance, and generally high, stable impedance;

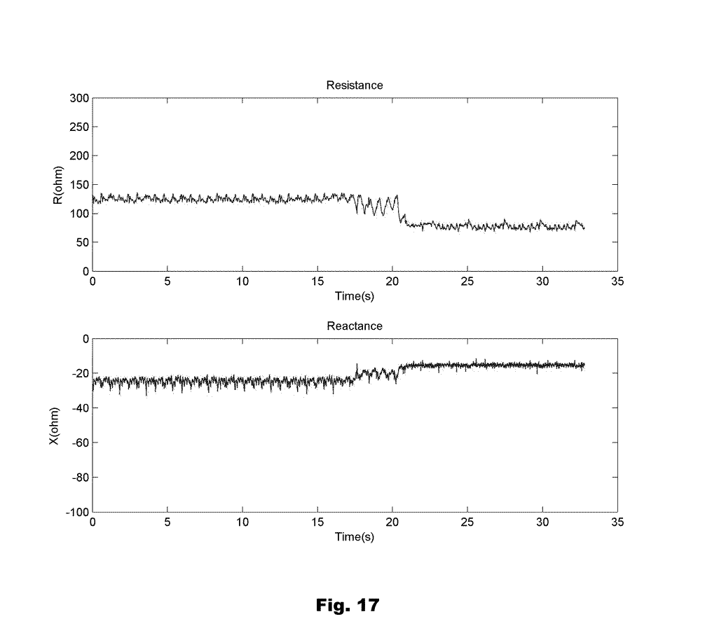

FIG. 17 is an in vivo unipolar resistance and impedance chart adjacent the LA lateral wall, compared to baseline (in the blood);

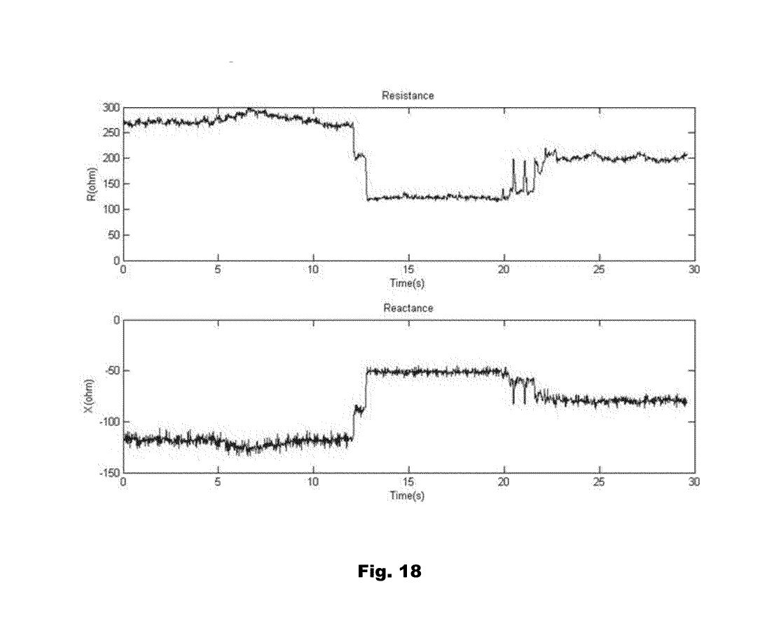

FIG. 18 is an in vivo unipolar resistance and impedance chart as the catheter moves from the pulmonary vein to the ostium to the pulmonary vein;

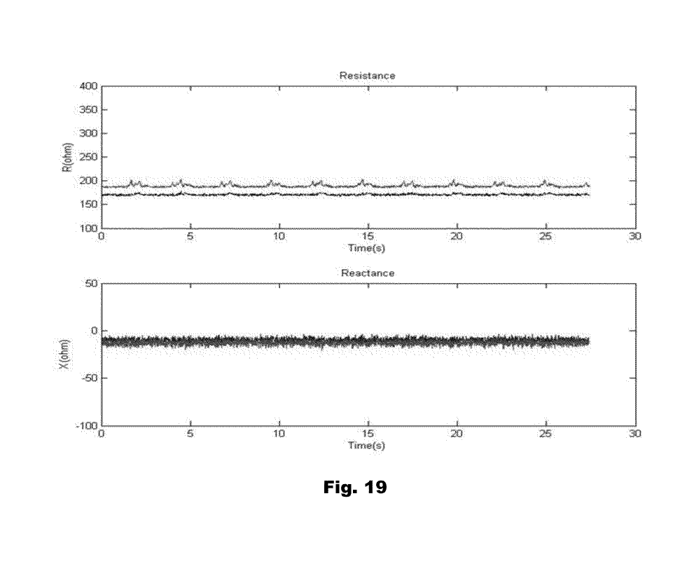

FIG. 19 is an in vivo two channel bipolar resistance and impedance chart in the blood pool;

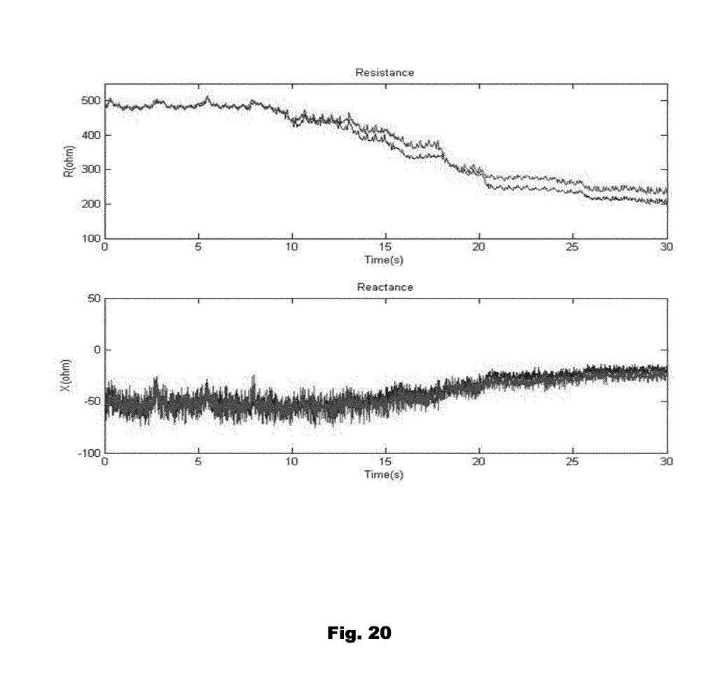

FIG. 20 is an in vivo two channel bipolar resistance and impedance chart as the catheter moves out of the pulmonary vein;

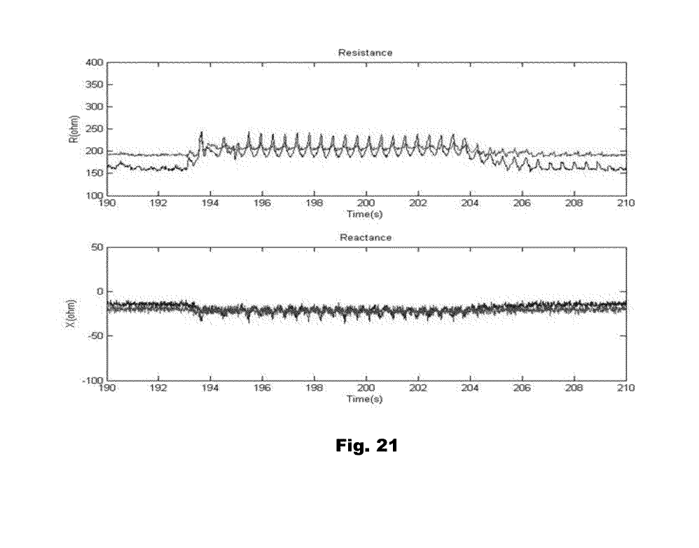

FIG. 21 is an in vivo two channel bipolar resistance and impedance chart in a vertical contact with the mitral valve as it is being retracted; and

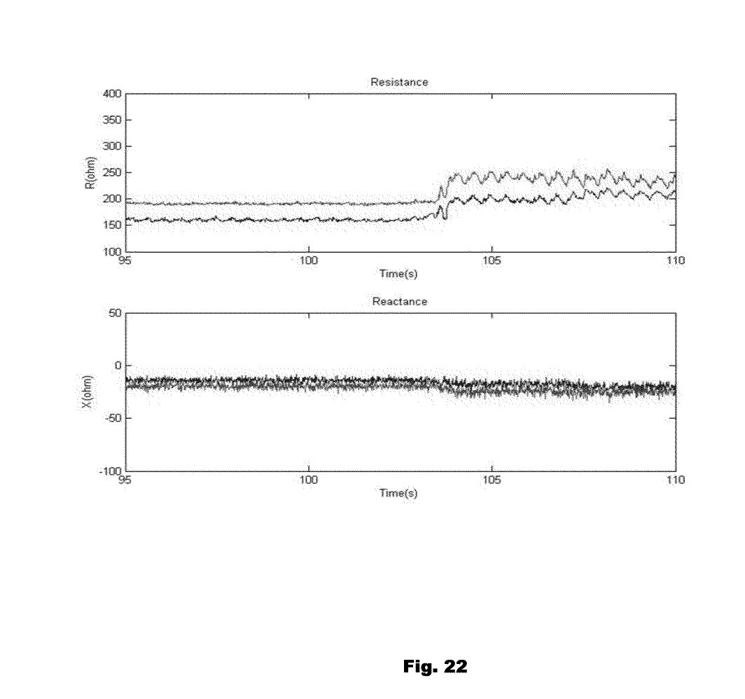

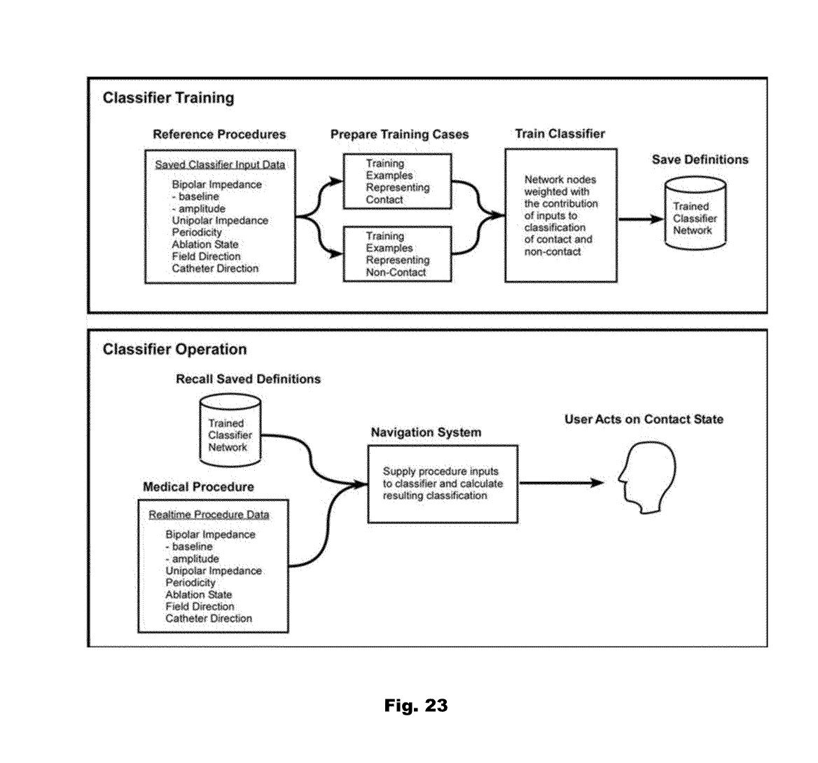

FIG. 22 is an in vivo two channel bipolar impedance chart in the in parallel contact with the mitral valve.

One preferred embodiment provides a method of determining the distance between an electrode catheter disposed in a body fluid adjacent an internal body surface, and the internal body surface. The method comprises applying an alternating voltage or an alternating current that alternates at between about 10 kHZ and about 100 kHz between at least one pair of electrodes on the electrode catheter, and determining the impedance between at least one pair of electrodes on the electrode catheter. The distance between the electrode catheter and the internal body surface is determined based at least in part on the determined impedance.

The impedance can be determined between the same pair of electrodes on the electrode catheter to which the alternating voltage or alternating current is applied.

In any of the embodiments described herein the electrode catheter can comprise a first pair of electrodes, and a second pair of electrodes disposed intermediate the first pair of electrodes. The alternating voltage or the alternating current can be applied to the first pair of electrodes, and the impedance can determined between the second pair of electrodes. The impedance is preferably determined between at least two pairs of electrodes on the electrode catheter, and the determined impedances are used to determine the distance between the electrode catheter and the internal body surface.

In any of the embodiments described herein, the tissue temperature can be determined, for example using unipolar impedance measurements from the tip electrode to a dispersive electrode. This temperature can be used either to correct or adjust the determined impedance, or it can be directedly as an input to the determination of the distance between the electrode catheter and the internal body surface.

The distance between the electrode catheter and the internal body surface can determined computationally, using an algorithm using the determined impedances as at least one input. Alternatively the distance between the electrode catheter and the internal body surface can be determined by a look-up table using the determined impedance. The look up table can be constructed computationally using an algorithm or model or finite-element simulations, or the look up table can be constructed experimentally, either in an environment generally representative of the specific environment, or in a series of calibrating measurements in the actual environment.

The look-up table in any of the embodiments described can use the difference of inter-electrode resistances between the determined values and a baseline value that corresponds to the same catheter being placed in the bodily fluid away from tissue surfaces. The baseline value can be determined from inter-electrode resistance measurements as at least one of the electrodes enters or leaves a sheath. The baseline value can also be determined by inter-electrode resistance measurements on a separate reference catheter placed within the same body fluid away from tissue surfaces. The baseline can be determined once, multiple times, or continuously. The base line can also be determined from measurements of the electrical conductivity of a sample of the body fluid. The baseline can also be determined from estimations of the electrical conductivity using a physiological model, which can take into account factors such as the amount of injected and ingested fluids over time, patient weight and kidney competence.

According to another embodiment, a method of determining the distance between an electrode catheter in a body fluid adjacent an internal body surface, and the internal body surface, includes determining the impedance between at least one pair of electrodes on the electrode catheter at an alternating voltage or an alternating current that alternates at between about 10 kHz and about 100 kHz, at at least two locations. The determined impedances from the at least two locations are used to determine the distance between the electrode catheter and the internal body surface.

The impedance is preferably determined between at least two pairs of electrodes on the electrode catheter. The distance between the electrode catheter and the internal body surface can be determined by a calculation using the determined impedances as an input or using a look up table.

According to another embodiment, a method of determining the orientation of an electrode catheter in a body fluid adjacent an internal body surface, relative to the internal body surface, comprises applying an alternating voltage or alternating current at between about 10 kHz and about 100 kHz, between at least two pairs of electrodes on the electrode catheter. The impedance between the at least one pair of electrodes on the electrode catheter is determined. The orientation of the electrode catheter relative to the internal body surface, is then determined using the determined impedances.

The impedance is determined between at least two pairs of electrodes on the electrode catheter, and these determined impedances are used to determine orientation of the electrode catheter relative to the internal body surface. The orientation can be determined by a calculation using the determined impedance as an input, or from a look-up table.

According to another embodiment, a method of determining the orientation of the electrode catheter in a body fluid adjacent an internal body surface, relative to the internal body surface comprises determining the impedance between at least one pair of electrodes on the electrode catheter at an alternating voltage or alternating current, alternating at between about 10 kHz and about 100 kHz at at least two locations. These determined impedances are used to determine the orientation of the electrode catheter relative to the internal body surface. The impedance is preferably determined between at least two pairs of electrodes on the electrode catheter, and these determined impedances are used to determine the orientation of the electrode catheter relative to the internal body surface. The orientation of the electrode catheter can be determined by a calculation using the determined impedances as an input or by using a look-up table and the measured impedances.

According to another embodiment of this invention, a method of estimating the contact force between the tip of an electrode catheter and the tissue surface with which it is making contact comprises using a local compliance model of the tissue which uses the negative distance and orientation of the catheter relative to the undeformed tissue surface as inputs.

According to another embodiment of this invention, method of determining catheter tip to body surface contact comprises using a classifier with a plurality of inputs including at least one bipolar impedance measurement at between about 10 kHZ and about 100 kHz and at least one unipolar impedance from the tip of the catheter. This classifier can comprise a an artificial neural network,

For magnetically navigated catheters, the difference in angle between a magnetically enabled catheter and a controlling magnetic navigation field as an input to the classifier. Additional or alternative inputs can comprise changes in the periodicity of the impedance signal as an input to the classifier.

According to another embodiment of this invention, a method for detection of catheter irrigation rate of an electrode catheter having a plurality of electrodes including a tip electrode, comprises detecting a change in capacitance component of the determined impedance between a first pair of electrodes that includes the tip, and a second pair of electrodes.

According to another embodiment of this invention, a method for determining the instant in time when a group of adjacent electrodes placed near the tip or on the shaft of a catheter exit from a sheath into a chamber of body fluid or retract from the chamber into the sheath, comprises measuring the impedance between pairs of the electrodes as the catheter moves to obtain a sequence of impedance changes, and matching the obtained pattern to a predetermined pattern.

According to another embodiment of this invention, a method of creating a parametric model representation of a local tissue surface method comprising representing local tissue surface with a parametric model. For a linear model, distance and two angles for orientation are used. For a quadratic model, distance, two curvatures for shape, and two angles for orientation are used.

In some embodiments the results from 3D FEA and ex-vivo experiments are used to develop a functional model that estimates impedance between two pairs of electrodes as a function of surface parameters.

In some embodiments of this invention a functional model that relates impedance to contact force is used.

In some embodiments of this invention, initial local surface parameters are estimated from previous localization measurements (e.g. Carto or NavX map) or previous 3D imaging (e.g. CT or MRI scan).

In some embodiments of this invention, a previously developed functional model, and a recursive, on-line parameter identification algorithm such as least-squares or maximum likelihood, are used and updated as new impedance measurements are obtained every measurement period (e.g. 10 ms)

In some embodiments the catheter position/orientation is changed to improve estimation.

In some embodiments the parameters are updated as the catheter makes contact with the tissue.

In some embodiments force is estimated using the previously developed functional model.

In some embodiments contact stability is determined from estimates of contact force, tissue distance and orientation change within a cardiac cycle.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The term “about” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters. For example, the terms “generally,” “about,” and “substantially,” may be used herein to mean within manufacturing tolerances. Or for example, the term “about” as used herein when modifying a quantity of an ingredient or reactant of the invention or employed refers to variation in the numerical quantity that can happen through typical measuring and handling procedures used, for example, when making concentrates or solutions in the real world through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about,” the claims include equivalents to the quantities.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method of determining the distance between an electrode catheter disposed in a body fluid adjacent an internal body surface, and the internal body surface, the method comprising:
applying an alternating voltage or an alternating current that alternates at between about 10 kHZ and about 100 kHz between at least one pair of electrodes on the electrode catheter;
determining the impedance between at least one pair of electrodes on the electrode catheter; and
determining the distance between the electrode catheter and the internal body surface based at least in part on the determined impedance.

2. The method according to claim 1 wherein the impedance is determined between the same pair of electrodes on the electrode catheter to which the alternating voltage or alternating current is applied.

3. The method according to claim 1 wherein the electrode catheter comprises a first pair of electrodes, and a second pair of electrodes disposed intermediate the first pair of electrodes, and wherein the method comprises applying the alternating voltage or the alternating current with the first pair of electrodes, and wherein the impedance is determined between the second pair of electrodes.

4. The method according to claim 1 further comprising determining tissue temperature using unipolar impedance measurements from the tip electrode to a dispersive electrode, and wherein the determination of the distance between the electrode catheter and the internal body surface is based at least in part on the determined impedance and the determined tissue temperature.

5. The method according to claim 1 wherein the impedance is determined between at least two pairs of electrodes on the electrode catheter, and wherein the determined impedances between the at least two pairs of electrodes on the electrode catheter are used to determine the distance between the electrode catheter and the internal body surface.

6. The method according to claim 1 wherein the distance between the electrode catheter and the internal body surface is determined by an algorithm using the determined impedance as one input.

7. The method according to claim 1 wherein the distance between the electrode catheter and the internal body surface is determined by a look-up table using the determined impedance.

8. The method according to claim 7 wherein the look-up table uses the difference of inter-electrode resistances between the determined values and a baseline value that corresponds to the same catheter being placed in the bodily fluid away from tissue surfaces.

9. The method according to claim 8. wherein the baseline value is determined by inter-electrode resistance measurements at the moment right after the catheter exiting a sheath or right before the catheter entering a sheath.

10. The method according to claim 8. wherein the baseline value is determined by inter-electrode resistance measurements on a separate reference catheter placed within the same body fluid away from tissue surfaces.

11. The method according to claim 8 wherein the baseline value is determined from measurements of the electrical conductivity of the body fluid by removing a fluid sample and using an external measurement apparatus and then using the measured fluid conductivity value as input to a mathematical function that returns the baseline resistance.

12. The method according to claim 8. wherein the baseline resistance is determined from estimations of the electrical conductivity of the body fluid using a physiological model of conductivity as a function of the amount of injected and ingested fluids over time, patient weight and kidney competence.

13. The method according to claim 8. wherein the look-up table uses the ratio of radiofrequency power that would be delivered to the tissue wall from the tip electrode to the power that would be delivered to body fluid if such power were applied, using a model of electrical transmission obtained from finite-element simulations or bench measurements.

14. A method of determining the distance between an electrode catheter in a body fluid adjacent an internal body surface, and the internal body surface, the method comprising:
determining the impedance between at least one pair of electrodes on the electrode catheter at an alternating voltage or an alternating current that alternates at between about 10 kHz and about 100 kHz at at least two locations; and
using the determined impedances from the at least two locations to determine the distance between the electrode catheter and the internal body surface.

15. The method according to claim 14 wherein the impedance is determined between at least two pairs of electrodes on the electrode catheter.

16. The method according to claim 14 wherein the distance between the electrode catheter and the internal body surface is determined by a calculation using the determined impedances as an input.

17. The method according to claim 14 wherein the distance between the electrode catheter and the internal body surface is determined using a look-up table and the determined impedances.

18. A method of determining the orientation of an electrode catheter in a body fluid adjacent an internal body surface, relative to the internal body surface, the method comprising:
applying an alternating voltage or alternating current at between about 10 kHz and about 100 kHz, between at least two pairs of electrodes on the electrode catheter;
determining the impedance between the at least one pair of electrodes on the electrode catheter; and
determining the orientation of the electrode catheter relative to the internal body surface, using the determined impedance.

19. The method according to claim 18 wherein the impedance is determined between at least two pairs of electrodes on the electrode catheter, and wherein the determined impedances between at least two pairs of electrodes on the electrode catheter are used to determine orientation of the electrode catheter relative to the internal body surface.

20. The method according to claim 19 wherein the orientation of the electrode catheter relative to the internal body surface is determined by a calculation using the determined impedance as an input.

21. The method according to claim 19 wherein the orientation of the electrode catheter relative to the internal body surface is determined using a look-up table and the determined impedance.

22. A method of determining the orientation of the electrode catheter in a body fluid adjacent an internal body surface, relative to the internal body surface, the method comprising:
determining the impedance between at least one pair of electrodes on the electrode catheter at an alternating voltage or alternating current, alternating at between about 10 kHz and about 100 kHz at at least two locations; and
using the determined impedance from the at least two locations to determine the orientation of the electrode catheter relative to the internal body surface.

23. The method according to claim 22 wherein the impedance is determined between at least two pairs of electrodes on the electrode catheter, and wherein the determined impedances between at least two pairs of electrodes on the electrode catheter are used to determine the orientation of the electrode catheter relative to the internal body surface.

24. The method according to claim 22 wherein the orientation of the electrode catheter relative to the internal body surface is determined by a calculation using the determined impedances as an input.

25. The method according to claim 22 wherein the orientation of the electrode catheter relative to the internal body surface determined using a look-up table and the measured impedances.

26. A method of estimating the contact force between the tip of an electrode catheter and the tissue surface with which it is making contact, the method comprising the step of using a local compliance model of the tissue which uses the negative distance and orientation of the catheter relative to the undeformed tissue surface as inputs.

27. A method of determining catheter tip to body surface contact, the method comprising the step of: using a classifier with a plurality of inputs including at least one bipolar impedance measurements at between about 10 kHZ and about 100 kHz and at least one unipolar impedance from the tip of the catheter.

28. A method according to claim 27 where the classifier comprises an artificial neural network.

29. A method according to claim 27 further comprising using the the difference in angle between a magnetically enabled catheter and a controlling magnetic navigation field as an input to the classifier.

30. A method according to claim 27 further comprising using changes in the periodicity of the impedance signal as an input to the classifier.

31. A method for detection of catheter irrigation rate of an electrode catheter having a plurality of electrodes including a tip electrode, the method comprising the steps of detecting a change in capacitance component of the determined impedance between a first pair of electrodes that includes the tip, and a second pair of electrodes.

32. A method for determining the instant in time when a group of adjacent electrodes placed near the tip or on the shaft of a catheter exit from a sheath into a chamber of body fluid or retract from the chamber into the sheath, the method comprising measuring the impedance between pairs of the electrodes as the catheter moves to obtain a sequence of impedance changes, and matching the obtained pattern to a predetermined pattern.
